(19) United States
(12) Patent Application Publication
(10) Pub. No.: US 2015/0024416 A1
(43) Pub. Date: Jan. 22, 2015

(54) CORRECTION METHOD FOR ESTIMATING FREE LIGHT CHAIN PRODUCTION

(71) Applicants: THE BINDING SITE GROUP LIMITED, West Midlands (GB); THE UNIVERSITY OF WARWICK, West Midlands (GB)

(72) Inventors: Stephen Harding, West Midlands (GB); Richard Hughes, West Midlands (GB); Anne Bevins, West Midlands (GB); Richard Keir, West Midlands (GB); Michael Chappell, West Midlands (GB); Neil Evans, West Midlands (GB); Colin Hutchinson, West Midlands (GB)

(73) Assignees: THE UNIVERSITY OF WARWICK, West Midlands (GB); THE BINDING SITE GROUP LIMITED, West Midlands (GB)

(21) Appl. No.: 14/380,074

(22) PCT Filed: Feb. 15, 2013
(86) PCT No.: PCT/GB2013/050362

§ 371 Date: Aug. 21, 2014

(30) Foreign Application Priority Data
Feb. 21, 2012 (GB) 1202964.1

(51) Int. Cl. G01N 33/68

(52) U.S. Cl. CPC G01N 33/6857; G01N 2800/50
USPC 435/7.92; 436/501; 435/287.2; 422/69; 702/23

(57) ABSTRACT

The invention provides a method of estimating free light chain production (FLC) in a subject comprising (i) determining an amount of FLC in a sample from the subject; and (ii) correcting the amount of FLC in the sample for FLC cleared from the source of the sample by glomerular filtration and by reticuloendothelial (RE) clearance.

The invention relates to methods of estimating free light chain (FLC) production in a subject and to methods of predicting whether a subject who has had a transplant is likely to have an infection or analysis of increased FLC production.

The Applicants have for many years studied free light chains as a way of assaying for a wide-range of monoclonal gammopathies in patients. The use of such free light chains in diagnosis is reviewed in detail in the book “Serum Free Light Chain Analysis, Sixth Edition (2008) A. R. Bradwell, ISBN 9780704427969”. Polyclonal abnormalities, where there is decreased or increased production of polyclonal antibodies in patients is also known.

Antibodies comprise heavy chains and light chains. They usually have a two-fold symmetry and are composed of two identical heavy chains and two identical light chains, each containing variable and constant region domains. The variable domains of each light-chain/heavy-chain pair combine to form an antigen-binding site, so that both chains contribute to the antigen-binding specificity of the antibody molecule. Light chains are of two types, κ and λ and any given antibody molecule has either light chain but never both. There are approximately twice as many κ as λ molecules produced in humans, but this is different in some mammals. Usually the light chains are attached to heavy chains. However, some unattached “free light chains” are detectable in the serum or urine of individuals. Free light chains may be specifically identified by raising antibodies against the surface of the free light chain that is normally hidden by the binding of the light chain to the heavy chain. In free light chains (FLC) this surface is exposed, allowing it to be detected immunologically. Commercially available kits for the detection of κ or λ free light chains include, for example, “Freelite™”, manufactured by The Binding Site Limited, Birmingham, United Kingdom. The Applicants have previously identified that measuring the amount of free κ, free λ and/or free κ/free λ ratios, allows the detection of monoclonal gammopathies in patients. It has been used, for example, as an aid in the diagnosis of intact immunoglobulin multiple myeloma (MM), light chain MM, non-secretory MM, AL amyloidosis, light chain deposition disease, smouldering MM, plasmacytoma and MGUS (monoclonal gammopathies of undetermined significance). Detection of FLC has also been used, for example, as an aid to the diagnosis of other B-cell dyscrasia and indeed as an alternative to urinary Bence Jones protein analysis for the diagnosis of monoclonal gammopathies in general.

The Applicants have also previously identified that assaying for FLC can be used to predict long-term survival of individuals, even when the individual is an apparently healthy subject. (WO 2011/021041). They found that total FLC concentration is statistically, significantly linked to long-term survival. Moreover, this link appears to be similar or better than the link for existing long-term survival prognostic markers such as cholesterol, creatinine, and C-reactive protein. Assays for total FLC measurement are disclosed in the document. Assays measuring total FLC are available from The Binding Site, Birmingham, United Kingdom under the trademark “Combylite”.

Polyclonal antibody associated diseases, where more than one specific antibody has increased or decreased production are generally known. For example, this may be characterised by a general increase in antibody production or by two or more monoclonal antibodies, from two separate tumour sources, being present. Chronic infections, autoimmune diseases and many tumours cause increases in polyclonal immunoglobulins. Skin, pulmonary and gut diseases are more likely to cause increases to IgA concentrations, whilst systemic infections will increase all immunoglobulins, but especially IgG.

The amount of FLC identified in a sample of, for example serum or plasma, will be affected by a number of factors. FLCs are produced by B-cell lineage cells. Polyclonal FLC production can increase due to infections and acute and chronic inflammation. FLCs are cleared in healthy subjects almost entirely by renal clearance through the kidneys. However, in subjects with renal impairment clearance via the reticuloendotelial (RE) network will have a greater effect.

Conventionally the estimated FLC production has been corrected by directly adjusting the serum free light chain level using the glomerular filtration rate (GFR). GFR is a measure of the rate at which water and dissolved substances are filtered out of the blood per unit time via the kidney. Typical ways of calculating or estimating GFR use compounds injected into the subject which are known to be filtered by the glomerulus and not absorbed or secreted by renal tubules. These include Cr-EDTA and inulin to calculate GFR. Naturally occurring endogenous compounds such as creatinine and cystatin C have also been used to estimate GFR.

Hutchinson et at (Clin, J. A. Soc. Nephrol (2008), 3, 1684-1690) studied the quantitative assessment of serum and urinary polyclonal FLC in chronic kidney disease and CKD patients. This was used to assess the CKD stage. Samples of serum and urine were analysed for creatinine and cystatin C. The amount of creatinine was used to calculate an estimated GFR (eGFR) using for example the creatinine-based Cockroft-Gault equation. This was used to study the correlation of serum κ and FLC with eGFR in CKD patients. The paper notes that κ FLC is produced at approximately twice the rate of λ FLC, but λ FLC more frequently forms dimers which slow their renal clearance. Moreover, as renal clearance is reduced in CKD patients the RE system becomes increasingly important. RE is not influenced by the molecular weight of the FLC.

This shows that the relationship between FLC clearance and GFR is not directly proportional and that, to produce an accurate assessment of FLC production in a subject, the effect of RE clearance needs to be considered.

The Applicant has identified that correcting the observed FLC concentration for GFR factor and RE clearance improves the estimation of FLC production allowing better analysis of the data and diagnosis and prognosis of patients. This has utilised the realisation that the correction can be better estimated by fitting a model to datasets of data from individuals with varying GFR values, but who are otherwise normal or apparently healthy.

The invention provides a method of estimating free light chain production (FLC) in a subject comprising:

(i) determining an amount of FLC in a sample from the subject; and

(ii) correcting the amount of FLC in the sample for FLC cleared from the source of the sample by glomerular filtration and by reticuloendothelial (RE) clearance.

The glomerular filtration may be estimated for the subject, for example by using creatinine or cystatin C. Typically this uses the Cockroft-Gault equation (Cockroft D. W. and Gault M. H, Nephron. (1976) 16 31-41) or the creatinine based MDRD equation (Vervoort G. et at Nephrol. Dial. Transplant (2002) 17 (11) 1909-13).

GFR and/or RE may be measured. The RE correction value may be a predetermined constant.

The FLC may be λ FLC, κ FLC or the total FLC (λ+κ). GFR varies from λ to κ, hence the GFR may be corrected for the type of FLC measured in the sample. λ and κ may be determined separately and optionally added together to determine total FLC production. Alternatively total FLC may be measured using, for example, combined anti-FLC antibodies.

Typically the amount of FLC produced is estimated adding the corrected GFR to a RE correction factor and multiplying that total by amount of FLC determined in the sample to produce an estimate of FLC production in the subject.

The sample may be urine, but is typically blood, plasma or serum.

Typically the estimated FLC production is determined using the following formula:

$$P = FLC ( α GFR + {K}_{re} + \frac{{K}_{re}}{v} )$$

where P=the estimated FLC produced by the subject

FLC=the amount of FLC determined in the sample

α=a GFR correction factor for the type of FLC

GFR=the GFR determined for the subject

Kᵣₑ=an RE constant

ν=the ratio between the vascular plasma volume and the extravascular fluid volume

Kᵣₑ may be assumed steady for this model it may be assumed constant (typically at 1.6×10⁻⁴ min⁻¹). Alternatively, an RE variable may be determined for the model.

α^{κ} is typically 0.000420011, α^{λ} is typically 0.00038141. However, these may vary, for example, to take account of population differences.

ν is determined this way because RE clearance occurs in both places, but GFR is in the vascular space. The typical value for an average person and typically used in the equation is approximately 0.21

This may be used at step (ii) of the invention.

A computer implemented method comprising the use of a method according to the invention is also provided, wherein step (ii) and/or the formula is carried out in a computer.

An apparatus comprising a computer processor and memory, configured to carry out a method of the invention is provided. Assay devices for detecting an amount of FLC in a sample comprising such an apparatus is also provided. Assay devices for determining FLC are generally known in the art. It may comprise an output, such as a screen, to display the estimated amount of FLC produced.

The invention also provides a computer readable medium product comprising code which when executed on a computer causes a computer to execute a method of the invention.

The measurements of the FLC in the sample may be determined using methods generally known in the art.

Antibodies, or fragments of antibodies, specific for κ or λ FLC are generally known and are commercially available under the trade name Freelite™

Typically the FLC is determined by immunoassay, such as ELISA assays or utilising fluorescently labelled beads, such as Luminix™ beads.

ELISA, for example uses antibodies to detect specific antigens. One or more of the antibodies used in the assay may be labelled with an enzyme capable of converting a substrate into a detectable analyte. Such enzymes include horseradish peroxidase, alkaline phosphatase and other enzymes known in the art. Alternatively other detectable tags or labels may be used instead of, or together with, the enzymes. These include radioisotopes, a wide range of coloured and fluorescent labels known in the art including fluorescein, Alexa fluor, Oregon Green, BODIPY, rhodamine red, Cascade Blue, Marina Blue, Pacific Blue, Cascade Yellow, gold; and conjugates such as biotin (available from, for example, Invitrogen Ltd, United Kingdom). Dye sols, metallic sols, chemiluminescent labels or coloured latex may also be used. one or more of these labels may be used in the ELISA assays according to the various inventions described therein, or alternatively in the other assays, labelled antibodies or kits described herein.

The construction of ELISA-type assays is itself well known in the art. For example, a “binding antibody” specific for the FLC is immobilised on a substrate. The “binding antibody” may be immobilised onto the substrate by methods which are well known in the art. FLC in the sample are bound by the “binding antibody” which binds the FLC to the substrate via the “binding antibody”.

Unbound immunoglobulins may be washed away.

In ELISA assays the presence of bound immunoglobulins may be determined by using a labelled “detecting antibody” specific to a different part of the FLC of interest than the binding antibody.

Flow cytometry may be used to detect the binding of the FLC of interest. This technique is well known in the art for, for example, cell sorting. However, it can also be used to detect labelled particles, such as beads and to measure their size. Numerous text books describe flow cytometry, such as Practical Flow Cytometry, 3^{rd} Ed. (1994), H. Shapiro, Alan R. Liss, New York, and Flow Cytometry, First Principles (2^{nd} Ed.) 2001, A. L. Given, Wiley Liss.

One of the binding antibodies, such as the antibody specific for FLC, is bound to a bead, such as a polystyrene or latex bead. The beads are mixed with the sample and the second detecting antibody. The detecting antibody is preferably labelled with a detectable label, which binds the FLC to be detected in the sample. This results in a labelled bead when the FLC to be assayed is present.

Labelled beads may then be detected via flow cytometry. Different labels, such as different fluorescent labels may be used for, for example, the anti-free λ and anti-free κ antibodies. Other antibodies specific for other analytes described herein may also be used in this or other assays described herein to allow the detection of those analytes. This allows the amount of each type of FLC bound to be determined simultaneously or the presence of other analytes to be determined.

Alternatively, or additionally, different sized beads may be used for different antibodies, for example for different marker specific antibodies. Flow cytometry can distinguish between different sized beads and hence can rapidly determine the amount of each FLC or other analyte in a sample.

An alternative method uses the antibodies bound to, for example, fluorescently labelled beads such as commercially available Luminex™ beads. Different beads are used with different antibodies. Different beads are labelled with different fluorophore mixtures, thus allowing different analytes to be determined by the fluorescent wavelength. Luminex beads are available from Luminex Corporation, Austin, Tex., United States of America.

Lateral flow devices may also be used.

Preferably the assay used is a nephelometric or turbidimetric method.

A further aspect of the invention provides a method of identifying a FLC production level in a subject with elevated FLC levels comprising the use of a method according to the first aspect of the invention. The method shows better analysis of production in such patients. The patient may have an infection, HIV, or a cancer such as monoclonal or polyclonal B-cell disease. The patient may have Chronic Lymphocytic Leukaemia or Hodgkin's lymphoma.

The Applicant has also assessed the validity of using the method of the invention by using it to study cohorts of FLC information from transplant recipients who have had renal transplant. This showed that lower levels of FLC showed an increased risk of the patient developing an infection over the next 30 days.

A further aspect of the invention provides a method of identifying whether an immune suppressed patient has a risk of developing significant infection (one requiring treatment with antibiotics and/or antiviral agents) infection comprising detecting an amount of free light chain (FLC) in a sample from the patient, wherein a lower amount of FLC is associated with an increased likelihood of the patient developing an infection.

Immune suppressed patients, including autoimmunity patients or organ transplant recipients, can be subject to increased risks of infection. The method provides a way of identifying those patients who might develop infections.

The organ transplanted may be a renal transplant. The total amount of FLC may be determined or alternatively κ FLC or λ FLC. The sample may be urine but is typically blood, plasma or serum. The FLC may be determined by a method as described above.

Typically a value of below a normal value of 0.45 to 0.92 mg/min more typically indicates an increased risk of developing an infection within the next 30 days.

The transplant patient typically does not present with symptoms of a B-cell associated disease, or a polyclonal B-cell disease.

Monoclonal B-cell diseases, include myeloma (such as intact immunoglobulin myeloma, light chain myeloma, non-secretory myeloma), an MGUS (monoclonal gammopathy of undetermined significance), AL amyloidosis, Waldenström's macroglobulinaemia, Hodgkin's lymphoma, follicular centre cell lymphoma, chronic lymphocytic leukaemia, mantle cell lymphoma, pre-B cell leukaemia or acute lymphoblastic leukaemia, polyclonal associated diseases include hypergammaglobulineamia or hypogammaglobulineamia.

The Applicants have shown that using FLC concentration it is possible to predict likely infections. This means that prophylactic drugs such as antibiotics may be provided or alternatively the amount of immunosuppressive drugs given to the patients reduced.

The invention will now be described by way of example only with reference to the following figures:

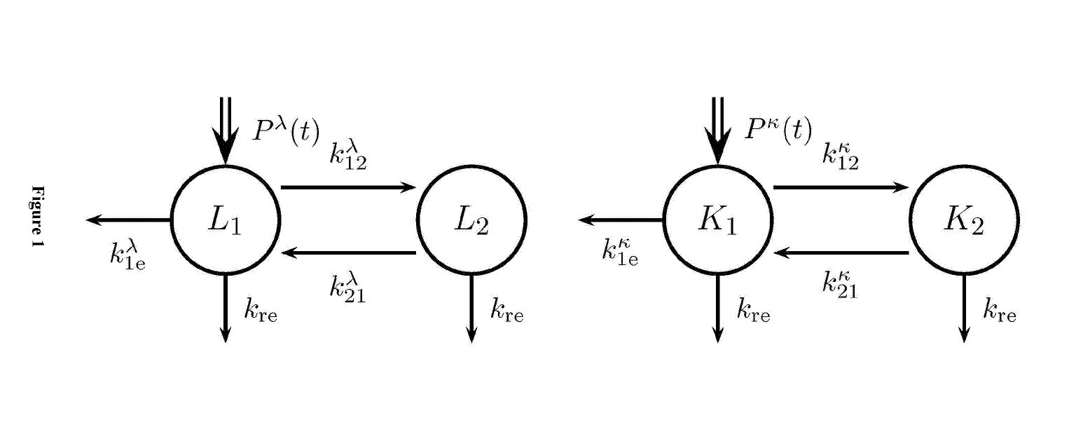

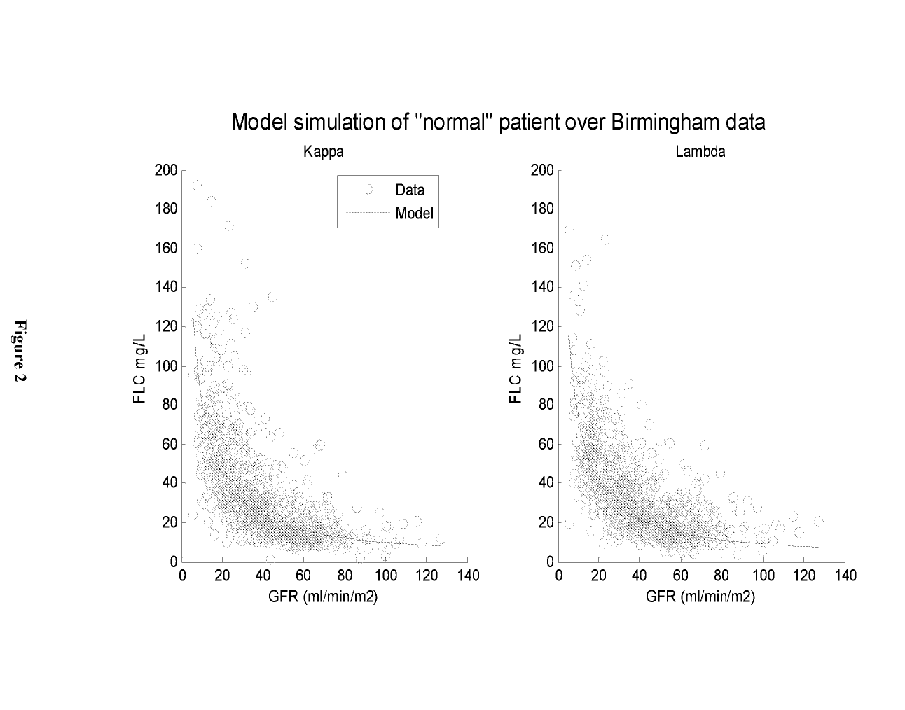

FIG. 1: shows models of FLC production and clearance for lambda and kappa.

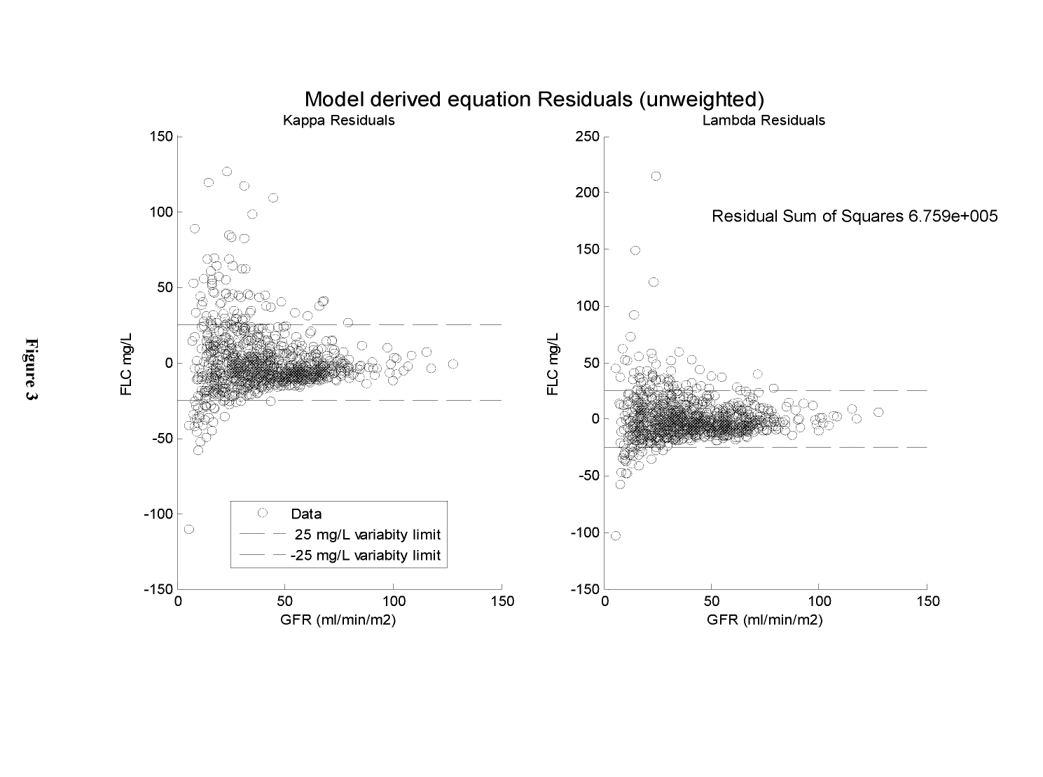

FIG. 2: shows a fitted model for λ and κ against patient data obtained for healthy patients for kappa and lambda.

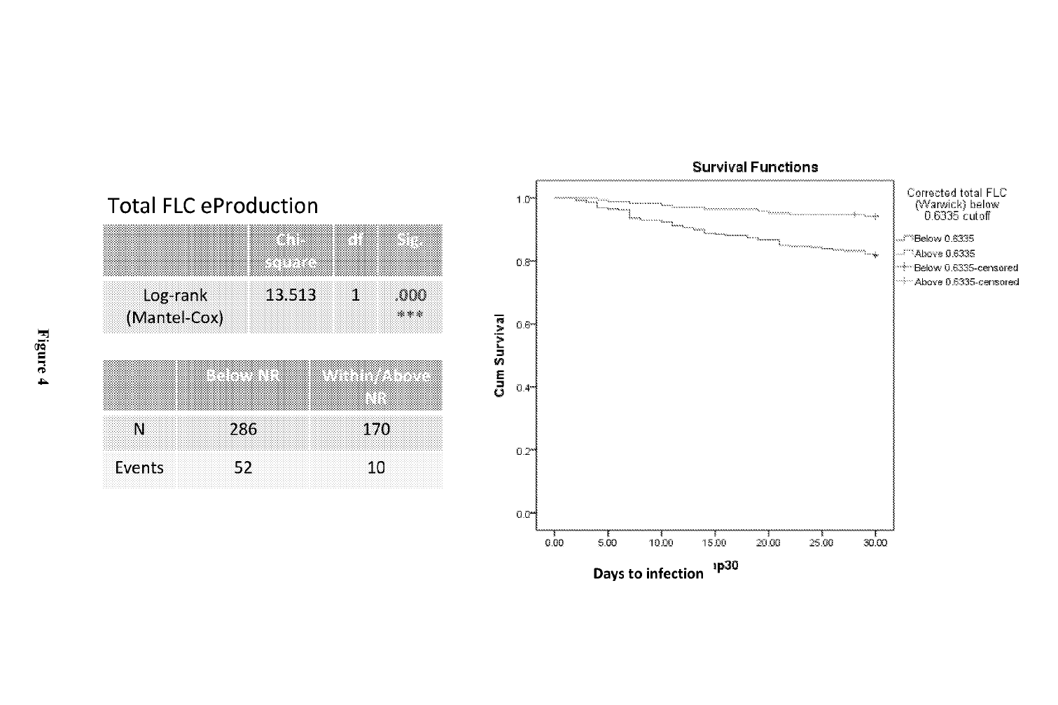

FIG. 3: shows fitted model residuals for the data set and fit shown in FIG. 2.

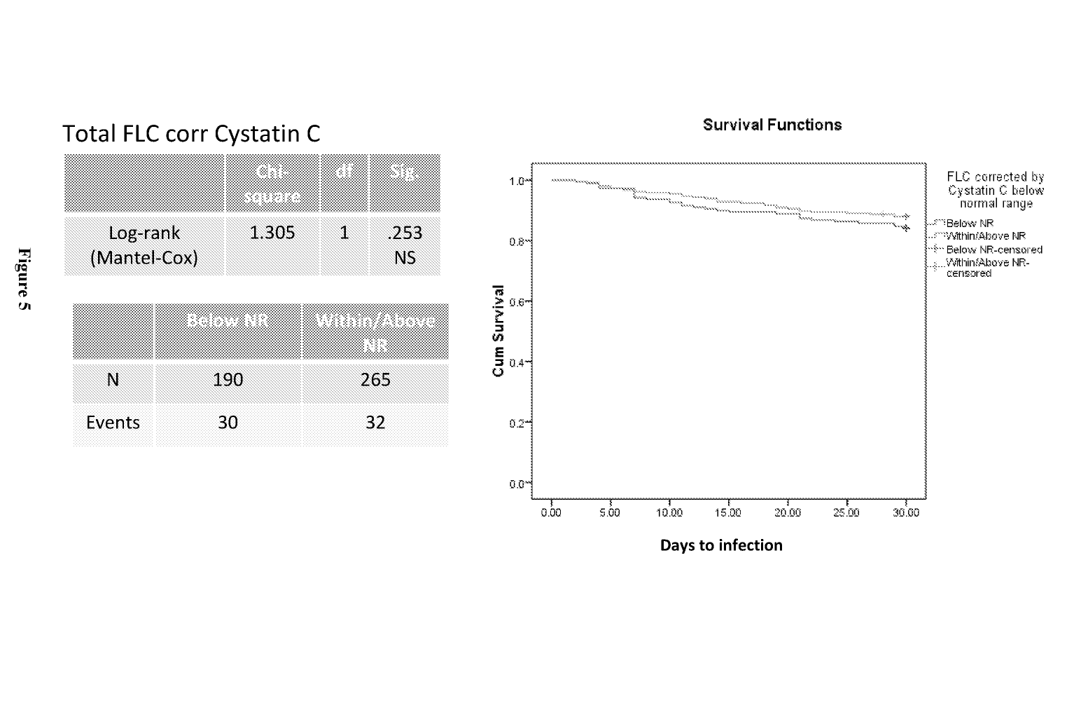

FIG. 4: shows analysis of survival over time for renal transplant patients with FLC production calculated by the equation of the invention (top line greater than 0.6335 mg/min, bottom line below 0.6335 mg/min).

FIG. 5: shows the same information but with FLC production corrected using Cystatin C eGFR calculation (lower line less than 15.3, upperline above 15.3).

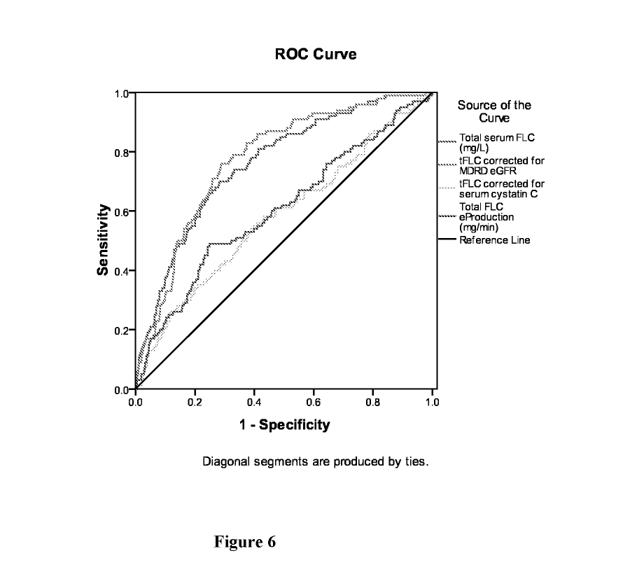

FIG. 6: shows ROC analysis of data from CKD patients for FLC levels, corrected FLC levels and estimated FLC production, 1-specificity at 0.3 the lines are (i) total FLC corrected for MDRD eGFR, total FLC (mg/L), estimated FLC production (mg/min), tFLC corrected for serum cystatin C, reference line.

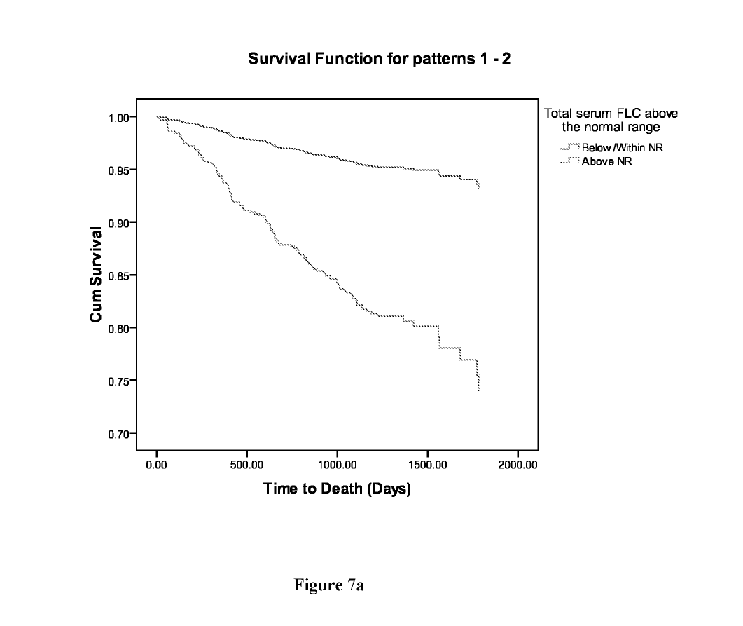

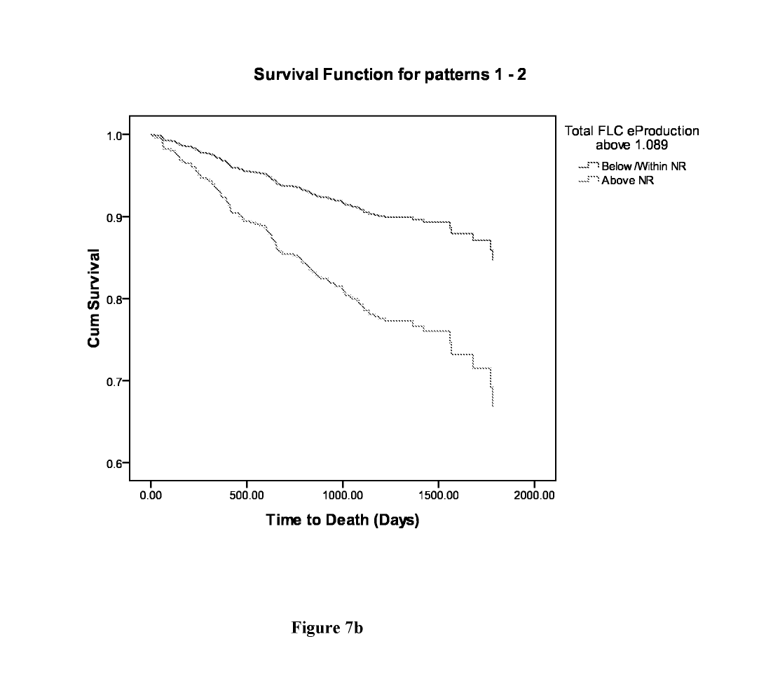

FIGS. 7a and 7b show Kaplan Meier survival curves for chronic kidney disease patients for a) total FLC [44.27] mg/L and (b) estimated FLC production.

### MODEL PRODUCTION

The Applicants identified that calculating the clearance of FLC requires the consideration of a number of parameters: GFR adjusted for κ or λ as it varies for each type of FLC. This is summarised in FIG. 1.

TABLE 1

| | | | Range (Standard | | |
|---|---|---|---|---|---|
| Parameter | Description | Relevant FLC | Deviation) | Base value | Units |
| P(t) | Indicated free light chain | κ | ±0.117 | 0.441 | mg min⁻¹ |
| | normal synthesis | λ | ±0.117 | 0.245 | |
| k₁ₑ | Normal renal clearance of | κ | (0.02064) | 0.0508 | min⁻¹ |
| | indicated free light chain | λ | (0.01032) | 0.00254 | |
| k₁₂ | Inter-compartment flow rates | κ | | 0.0122 | L min⁻¹ |
| and k₂₁ | (representing molecular flow | λ | | 0.0086 | L min⁻¹ |
| | around the body) | | | | |
| kᵣₑ | Reticuloendothelial clearance | | — | 1.6 × 10⁻⁴ | min⁻¹ |

The base values shown in Table 1 are from the literature or best estimate of available data.

This information was used to produce a model for steady rate renal function and constant FLC production over 24 hours. The amount of kappa found in plasma κ (κ_{Plas}^{ss}) or λ (λ_{Plas}^{ss}) is based on the following equation:

$${K}_{Plas}^{ss} = \frac{{vP}^{K} ( t )}{v ( {α}^{k} GFR + {k}_{re} ) + {k}_{re}}$$

$${λ}_{Plas}^{ss} = \frac{{vP}^{λ} ( t )}{v ( {α}^{λ} GFR + {k}_{re} ) + {k}_{re}}$$

ν=the ratio of volume between the vascular plasma volume and the extravascular fluid volume.

α=GFR correction value for λ and κ

P(T)=FLC synthesis overtime

Kᵣₑ=RE clearance

This was fitted over actual date of FLC versus GFR from a population set for patients with varying levels of GFR, though otherwise, by residual sum of squares analysis to show confidence in the equation and estimate the unknown parameters. The equation with values giving the lowest sum of squares is the one with greatest confidence.

FIGS. 2 and 3 show the fit to one such data set. This was repeated for several different data sets to improve the fit of the model (data not shown). Patients with C-reactive protein (CRP) levels above >10 mg/L were not included in the dataset to exclude patients showing signs of inflammation and infection.

The equation can then be applied to estimate production levels for kappa and lambda FLCs given the patients GFR and FLC concentrations.

$${P}^{K} ( t ) = {K}_{Plas}^{ss} ( {α}^{k} GFR + {k}_{re} + \frac{{k}_{re}}{v} )$$

$${P}^{λ} ( t ) = {λ}_{Plas}^{ss} ( {α}^{λ} GFR + {k}_{re} + \frac{{k}_{re}}{v} )$$

This also allows total FLC production to be estimated.

Analysis of Transplant Data

The equations described above were used to study total FLC against the risk of significant infections in renal transplant patients.

Data was analysed using the new equation to estimate FLC production compared to total FLC production corrected by prior art eGFR estimation using cystatin C.

FIG. 4 shows infection against survival for total estimated FLC production above 0.6335 mg/min total estimated FLC production (top line) and below 0.6335 mg/min bottom line. This shows that FLC may be used to indicate the likelihood of a patient having a significant infection in the next 30 days, opening up the possibility of further prophylactic treatment of these patients.

FIG. 5 shows the same data but calculated using the prior art cystatin C eGFR calculation to calculate the total FLC production. The difference in the two populations is significantly more difficult to see and not shown to be statistically significant using Kaplan Meier analysis. This demonstrates that the new equation for calculating FLC production improves the detection of trends from data presented, resulting in improved accuracy of patients for FLC values.

The utility of the correction of the data to estimate FLC production may be expanded to include the calculation of the production of FLC in patients with high levels of FLC. CKD patients, for example, often have high levels of CKD. Higher levels of FLC are associated with mortality. This is shown in FIG. 6. The corrected estimated production curve separates the observed deaths from those associated with renal impairment and those associated with elevated FLC production. This allows improved analysis of the data and assessment of risk factors.

Multivariate analysis shows that FLC production can be separated from FLS kidney clearance to total FLC levels. CRP and cystatin C are independent of FLC levels.
i) Univariate analysis of factors associated with significant infections at 30 days (following low FLC production) in a renal transplant population using Cox regression with Enter output

| Variable | Category | Hazard Ratio (95% CI) | P-value |
|---|---|---|---|
| Age | — | 1.082 (1.064, 1.100) | <0.001 |
| Sex | Male | 1 | |
| | Female | 0.605 (0.418, 0.875) | 0.008 |
| Ethnicity | White | 1 | |
| | Black | 0.781 (0.364, 1.680) | 0.528 |
| | Asian | 0.608 (0.308, 1.201) | 0.152 |
| | Other | 0.756 (0.240, 2.384) | 0.633 |
| ACEI | No | 1 | |
| | Yes | 0.804 (0.560, 1.156) | 0.239 |
| IHD | No | 1 | |
| | Yes | 2.597 (1.688, 3.994) | <0.001 |
| MI/Angio | No | 1 | |
| | Yes | 2.313 (1.325, 4.040) | 0.003 |
| CVA | No | 1 | |
| | Yes | 3.363 (1.807, 6.257) | <0.001 |
| Systolic BP | — | 1.007 (1.000, 1.015) | 0.041 |
| Diastolic BP (Median) | — | 0.988 (0.974, 1.001) | 0.077 |
| ACR (Median) | — | 1.000 (0.999, 1.002) | 0.693 |
| ACR (log) | | 1.199 (0.979, 1.469) | 0.079 |
| Albumin | — | 0.958 (0.931, 0.987) | 0.004 |
| Calcium | — | 0.135 (0.051, 0.357) | <0.001 |
| Phosphate | — | 0.992 (0.952, 1.035) | 0.718 |
| Creatinine | — | 1.004 (1.003, 1.005) | <0.001 |
| Creatinine (Log) | — | 17.826 (8.524, 37.280) | <0.001 |
| MDRD-eGFR | — | 0.957 (0.945, 0.968) | <0.001 |
| hs-CRP | — | 1.008 (1.005, 1.012) | <0.001 |
| hs-CRP (log) | | 2.744 (1.996, 3.771) | <0.001 |
| Cystain C | — | 1.659 (1.475, 1.865) | <0.001 |
| tFLC (uncorrected) | | 1.077 (1.005, 1.009) | <0.001 |
| tFLC (uncorrected) >NR | No | 1 | |
| | Yes | 4.264 (2.523, 7.206) | <0.001 |
| tFLC eProduction | | 1.829 (1.377, 2.429) | <0.001 |
| tFLC eProduction >1.089 | No | 1 | |
| | Yes | 2.422 (1.699, 3.453) | <0.001 |

ii) Multivariate analysis of factors associated with infection using Cox regression with a backwards selection procedure. The multivariate analysis indicated that Sex, WBC count, IgG level and total FLC eProduction were significantly associated with infection at 30 days. No other factors were found to be significant.

| Variable | Category | Hazard Ratio (95% CI) | P-value |
|---|---|---|---|
| Sex | Female | 3.078 (1.695, 5.590) | <0.001 |
| WBC count <5 | Yes | 3.762 (1.558, 9.083) | 0.003 |
| IgG <normal range | Yes | 2.873 (1.156, 7.140) | 0.023 |
| tFLC eProd <0.6335 | Yes | 2.933 (1.360, 6.326) | 0.006 |

Survival curves for total FLC production and estimated production are shown in FIGS. 7a and 7b.

1-21. (canceled)

22. A method of estimating free light chain production (FLC) in a subject comprising:
(i) determining an amount of FLC in a sample from the subject; and
(ii) correcting the amount of FLC in the sample for FLC cleared from the source of the sample by glomerular filtration and by reticuloendothelial (RE) clearance.

23. The method of 22, wherein a glomerular filtration rate (GFR) is estimated for the subject.

24. The method of claim 23, wherein the estimated GFR is corrected for the type of FLC determined in the sample to produce a correct GFR.

25. The method of 22, wherein the FLC is λ FLC, κ FLC or total FLC.

26. The method of 22, wherein the amount of FLC produced is estimated by adding the corrected GFR to a RE correction factor and multiplying that total with the amount of FLC determined in the sample to produce an estimate of FLC production in the subject.

27. The method of 22, wherein the sample is a sample of blood, plasma or serum.

28. The method of 22, wherein the estimated FLC production is determined using the following formula:
$$P = FLC ( α GFR + {K}_{re} + \frac{{K}_{re}}{v} )$$
where P=the estimated FLC produced by the subject (mg/min)
FLC=the amount of FLC determined in the sample (mg/L)
a=a GFR correction factor for the type of FLC
GFR=the GFR estimated for the subject
Kᵣₑ=an RE constant, and
ν=the ratio of volume between the vascular plasma volume and the extravascular fluid volume.

29. The method of claim 22 wherein the subject has elevated FLC levels.

30. A computer implemented method comprising the method of claim 22, wherein step (ii) and/or the use of the formula is carried out on a computer.

31. An apparatus comprising a computer processor and memory, configured to carry out the method of claim 22.

32. An assay device for determining the level of FLC production in a subject by quantifying the amount of FLC in a sample comprising the apparatus of claim 31.

33. A method of identifying whether an immune suppressed patient has a risk of developing an infection comprising detecting an amount of free light chain (FLC) in a sample from the patient, wherein a lower amount of FLC is associated with an increased likelihood of the patient developing an infection.

34. The method of claim 33, wherein the patient is an organ transplant recipient.

35. The method of claim 33, wherein the amount of free light chain is the amount of total free light chain in the sample.

36. The method of claim 33, wherein the risk of infection is likely to occur within 90 days, 60 days or within 30 days of taking the sample from the patient.

37. The method of claim 33, wherein FLC is determined in a sample of serum from the subject.

38. The method of claim 33, wherein the total FLC is determined by immunosassay using anti-free light chain antibodies.

39. The method of claim 35, wherein the antibodies are a mixture of anti-free κ light chain and anti-free λ light chain antibodies.

40. The method of claim 33, wherein the method comprises detecting the amount of FLC by nephelometry or turbidimetry.

41. The method of claim 33, wherein the subject does not have symptoms of B-cell associated disease.

42. A computer readable medium product comprising code, which when executed on a computer causes a computer to execute the method of claim 22.
